(19) United States
(12) Patent Application Publication
(10) Pub. No.: US 2026/0114921 A1
(43) Pub. Date: Apr. 30, 2026

(54) ADJUSTABLE CATHETERS, SYSTEMS, AND METHODS FOR FORMING A FISTULA

(71) Applicant: TVA MEDICAL, INC., Franklin Lakes, NJ (US)

(72) Inventors: Andrzej J. CHANDUSZKO, Franklin Lakes, NJ (US); Hoang P. NGUYEN, Franklin Lakes, NJ (US); Kristin N. ROMINGER, Franklin Lakes, NJ (US); Breanna Simpson, Franklin Lakes, NJ (US); Mark S. THOMAS, Franklin Lakes, NJ (US)

(21) Appl. No.: 19/471,501

(22) PCT Filed: Apr. 28, 2023
(86) PCT No.: PCT/US2023/020373

§ 371 Date: Oct. 1, 2025

(51) Int. Cl. A61B 18/14; A61B 17/11; A61M 25/00

(52) U.S. Cl. CPC A61B 18/1492; A61B 2017/1107; A61B 2018/1475; A61M 2025/0096

(57) ABSTRACT

A system for forming a fistula including a first catheter and a second catheter. The first and second catheters are configured to be advanced in a first vessel and a second vessel, respectively. The first catheter includes a first treatment portion defining a first treatment slot and an electrode advanceable from the first treatment slot. The second catheter includes a second treatment portion defining a second treatment slot configured to receive the electrode. The length of the first and second treatment slots are also adjustable.

### TECHNICAL FIELD

The present disclosure relates to catheters and systems for endovascular treatments of a blood vessel, and more particularly catheters and systems for aligning catheters with adjustable electrode housings for forming an adjustable length fistula or providing other endovascular treatment.

### BACKGROUND

Endovascular treatments of a blood vessel may include fistula formation. A fistula is generally a passageway formed between two internal organs (e.g., blood vessels or other bodily organs). Forming a fistula between two blood vessels can have one or more beneficial functions. For example, the formation of a fistula between an artery and a vein may provide access to the vasculature for hemodialysis patients. Specifically, forming a fistula between an artery and a vein allows blood to flow quickly between the vessels while bypassing the capillaries. In other instances, a fistula may be formed between two veins to form a veno-venous fistula. Generally, arterio-venous fistula formation requires surgical dissection of a target vein, and transecting and moving the vein for surgical anastomosis to the artery. It may therefore be useful to find less invasive and reliable devices and methods for aligning blood vessels and forming a fistula between the aligned blood vessels. It may also be useful to provide a device that is capable of forming various sized fistulas.

### SUMMARY

One challenging aspect of forming a fistula between blood vessels is related to the size and flow through the fistula itself. High flow fistulas may be associated with negative cardiovascular outcomes such as heart failure. Too low of fistula flow may also negatively impact fistula maturation in the case of hemodialysis, possibly resulting in fistula maturation failure. Additionally, in the treatment of hypertension, too low of a fistula flow rate may be unable to sufficiently lower blood pressure. Embodiments of the present disclosure are directed to systems and catheters for fistula formation that allow for control over the length of the fistula, and thereby flow rates therein, by the use of an adjustable electrode housing, as will be described in greater detail below. This may allow a single system to be used for a variety of locations, applications, and anatomies.

In one embodiment, a system for forming a fistula includes a first catheter and a second catheter. The first and second catheters are configured to be advanced in a first vessel and a second vessel, respectively. The first catheter includes a first treatment portion defining a first treatment slot and an electrode advanceable from the first treatment slot. The second catheter includes a second treatment portion defining a second treatment slot configured to receive the electrode. The length of the first treatment slot is adjustable, and the length of the second treatment slot may be adjustable or non-adjustable.

In another embodiment, a catheter for forming a fistula includes a catheter body, an electrode housing coupled to the catheter, and an electrode. The electrode housing includes a first portion and a second portion, wherein the first portion and the second portion together define a treatment region extending between a first end wall of the first portion and a second end wall of the second portion. The first portion also defines a receiving slot positioned within the treatment region. Additionally, the second portion is slidably positioned within the receiving slot such that a distance between the first end wall and the second end wall is adjustable. The electrode is also configured to protrude from the treatment region.

In yet another embodiment a catheter for forming a fistula includes a treatment portion, an electrode configured to project from the treatment slot, and a sliding limit assembly. The treatment portion includes a first portion and a second portion, the treatment portion defining a treatment slot. The first portion is also adjustable relative to the second portion to adjust a length of the treatment slot. The sliding limit assembly 180 limits a distance the second portion travels relative to the first portion.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically illustrates a system of catheters for endovascular treatment of a blood vessel, according to one or more embodiments shown and described herein;

FIG. 2A schematically illustrates a perspective view of a catheter, according to one or more embodiments shown and described herein, without an electrode, for purposes of illustrating the first treatment portion;

FIG. 2B schematically illustrates a longitudinal cross-section of the catheter of FIG. 2A, according to one or more embodiments shown and described herein, with the electrode;

FIG. 2C schematically illustrates a top view of the catheter of FIG. 2B as viewed along the-Z direction of the depicted coordinate axes of FIG. 1, according to one or more embodiments shown and described herein;

FIG. 3A schematically illustrates a longitudinal cross-section of a catheter, according to one or more embodiments shown and described herein;

FIG. 3B schematically illustrates an exploded view of the outer and inner housings of the catheter of FIG. 3A, according to one or more embodiments shown and described herein;

FIG. 4A schematically illustrates a longitudinal cross-section of a catheter with first and second electrodes before exposing the second longitudinally offset electrode, according to one or more embodiments shown and described herein;

FIG. 4B schematically illustrates the catheter after exposing the second longitudinally offset electrode, according to one or more embodiments shown and described herein;

FIG. 5A schematically illustrates a longitudinal cross-section of a catheter, according to one or more embodiments shown and described herein, before adjusting the length of the treatment slot;

FIG. 5B schematically illustrates a longitudinal cross-section of the catheter of FIG. 5A, according to one or more embodiments shown and described herein, after adjusting the length of the treatment slot;

FIG. 5C schematically illustrates an exploded view of the catheter of FIG. 5A, according to one or more embodiments shown and described herein;

FIG. 6A schematically illustrates an exploded view of a catheter, according to one or more embodiments shown and described herein;

FIG. 6B schematically illustrates a longitudinal cross-section of the catheter of FIG. 6A, according to one or more embodiments shown and described herein;

FIG. 6C schematically illustrates another longitudinal cross-section of the catheter of FIGS. 6A-6B, according to one or more embodiments shown and described herein;

FIG. 6D schematically illustrates another longitudinal cross-section of the catheter of FIGS. 6A-6C, according to one or more embodiments shown and described herein;

FIG. 7A schematically illustrates an exploded view of a catheter, according to one or more embodiments shown and described herein;

FIG. 7B schematically illustrates a longitudinal cross-section of the catheter of FIG. 7A, according to one or more embodiments shown and described herein;

FIG. 7C schematically illustrates another longitudinal cross-section of the catheter of FIGS. 7A-7B, according to one or more embodiments shown and described herein;

FIG. 8A schematically illustrates an exploded view of a catheter, according to one or more embodiments shown and described herein;

FIG. 8B schematically illustrates a longitudinal cross-section of the catheter of FIG. 8A, according to one or more embodiments shown and described herein;

FIG. 8C schematically illustrates another longitudinal cross-section of the catheter of FIGS. 8A-8B, according to one or more embodiments shown and described herein;

FIG. 9A schematically illustrates a perspective view of a catheter, according to one or more embodiments shown and described herein, before adjusting the length of a treatment slot.

FIG. 9B schematically illustrates a perspective view of a catheter, according to one or more embodiments shown and described herein, after adjusting the length of the treatment slot.

FIG. 10A schematically illustrates a perspective view of a catheter with an interlocking mechanism, spring, and a compression element for freeing the interlocking mechanism, according to one or more embodiments shown and described herein.

FIG. 10B schematically illustrates a perspective view of a catheter with an interlocking mechanism, spring, hinge, and a compression element for freeing the interlocking mechanism, according to one or more embodiments shown and described herein.

FIG. 10 schematically illustrates a handle of a catheter such as any described herein having one or more user input devices for controlling the size of a resulting fistula, according to one or more embodiments shown and described herein.

FIG. 11 illustrates a flow chart depicting a method for forming a fistula, according to one or more embodiments shown and described herein.

FIG. 13A schematically illustrates a first catheter of the system of FIG. 1 being advanced through a first blood vessel and a second catheter of the system of FIG. 1 being advanced through a second vessel, according to one or more embodiments shown and described herein;

FIG. 13B Schematically illustrates treatment of the first blood vessel and the second blood vessel with the first catheter and the second catheter of FIG. 10A, according to one or more embodiments shown and described herein; and

FIG. 13C schematically illustrates a fistula formed between the first blood vessel and the second blood vessel, according to one or more embodiments shown and described herein.

Reference will now be made in greater detail to various embodiments of the present disclosure, some embodiments of which are illustrated in the accompanying drawings. Whenever possible, the same reference numerals will be used throughout the drawings to refer to the same or similar parts.

### DETAILED DESCRIPTION

Embodiments described herein are directed to devices, systems, and methods for endovascular treatment of a blood vessel such as, but not limited to forming a fistula, wire crossing procedures, bypass procedures, etc. For example, a catheter may be placed in each of two adjacent blood vessels to form a fistula therebetween with the catheters. However, as previously stated, the flow rate is determined largely by the size of the fistula. Accordingly, depending on the desired outcome, it is desirable to control the size of the resulting fistula. The needed size of the fistula can be dependent on a variety of factors, including but not limited to: subject anatomy or physiology, intended anatomical treatment (hemodialysis or hypertension treatment), and anatomical location of the fistula. Accordingly, embodiments herein provide systems and catheters for forming fistulas of varied sizes that provide flexibility in use for many applications. The catheter is also capable of being user controlled and sized before and/or after entry of the catheter into the subject. These and additional features and benefits will be described in greater detail herein.

As used herein, the term “proximal” means closer to or in the direction of an origin of an element, such as a catheter. The origin of a catheter may be a handle or other user-manipulated portion of the catheter. The term “distal” means further from the origin, or handle, of the catheter. Put another way, the term “distal” means closer to or in the direction of a tip of a catheter, which is separated from a handle or other user-manipulated portion of the catheter by the length of the catheter body.

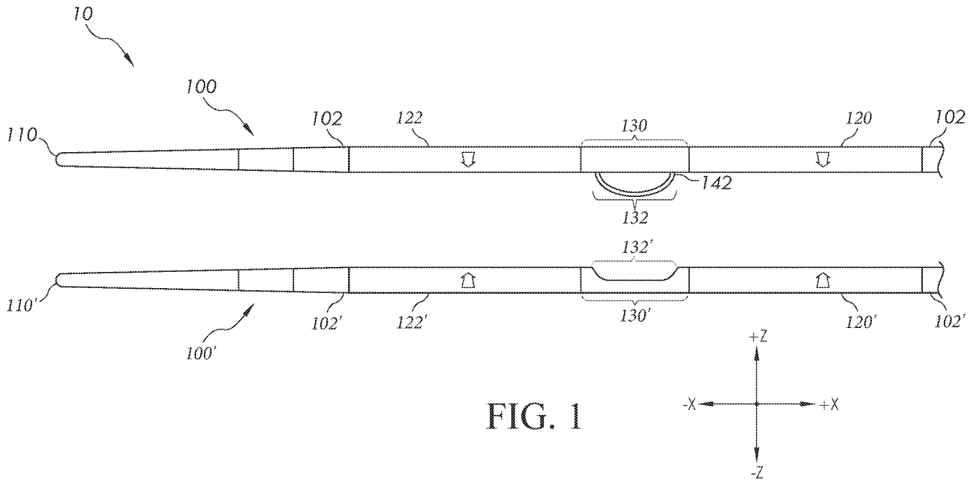

Referring now to FIG. 1, a system 10 for providing endovascular treatment of a blood vessel, such as fistula formation, is schematically depicted. The system 10 may include a first catheter 100 and a second catheter 100′. It may be appreciated that the first catheter 100 and the second catheter 100′ are substantially similar to one another. Accordingly, description of the first catheter 100 generally applies to the second catheter 100′ unless otherwise noted or apparent. It is noted that the first catheter 100 and the second catheter 100′ may be provided within a kit and/or separately from one another.

The first catheter 100 may include a catheter body 102 and a treatment portion 130, which may also be referred to herein as a “first treatment portion”. It is noted that the first catheter 100 may include a greater or fewer number of components without departing from the scope of the present disclosure.

The catheter body 102 may be sized to be advanced through a blood vessel and may include a distal tip 110 that is may be shaped and/or sized to aid in advancement of the first catheter 100 through a blood vessel. For example, the distal tip 110 may be pointed, tapered, and/or atraumatic for advancement through a blood vessel. The catheter body 102 may have any cross-sectional shape and any diameter suitable for intravascular use. The first catheter 100 may include or define one or more passageways (not shown) extending at least partially along or through the catheter body 102, the treatment portion 130, or both. For instance, the one or more passageways may extend at least partially longitudinally through the catheter body 102 and the treatment portion 130 in the direction of an x-axis of the depicted coordinate axes of FIG. 1. The catheter body 102 and/or the treatment portion 130 may be formed of any material or combination of materials able to be traversed through a vasculature of a body. For example, the catheter body 102 and/or the treatment portion 130 may include, silicone, rubber, etc. The catheter body 102 and/or the treatment portion 130 may be formed through any material generation process, including but not limited to injection molding or other machining process.

As noted above, the first catheter 100 may have the treatment portion 130 for endovascular treatment of a blood vessel. In embodiments, the treatment portion 130 may define a treatment slot 132, which may also be referred to herein as a “first treatment slot,” and an electrode 142, such as a leaf spring electrode, having an exposed ablation surface. The electrode 142 may extend through the one or more passageways, and thereby through the catheter body 102 and the treatment portion 130. The electrode 142 may be advanceable from the first treatment slot 132, wherein a length of the first treatment slot is adjustable, as discussed in further detail herein. The electrode 142 may be coupled to a power source, such as via a lead wire or other conductor attached thereto. The electrode 142 may, in some embodiments, be coupled to a push/pull rod 182, which may additionally function as a lead wire or other conductor. When activated, current may be supplied to and/or carried from tissue and fluid via the ablation surface to facilitate ablation or vaporization of tissue to form a fistula. As illustrated in FIG. 1, the electrode 142 may be arc shaped, though other shapes are contemplated and possible (e.g., rectangular, square, angular, etc.).

In embodiments, the first catheter 100 may further include one or more magnetic arrays for alignment or coaptation purposes of the first catheter 100 to the second catheter 100′. For example, and in embodiments, the first catheter 100 may include a proximal magnetic array 120 and a distal magnetic array 122.

The proximal magnetic array 120 may be arranged on or within the catheter body 102 proximal to and adjacent the treatment portion 130. The proximal magnetic array 120 may include a plurality of magnetic elements arranged in a longitudinal array along a length of the catheter. Each of the magnetic elements of the plurality may have substantially the same dimensions (e.g., height, width, and depth) and substantially the same magnetic strength. In some embodiments, the plurality of magnetic elements may have a combination of different sized magnets, different shaped magnets, and/or magnets of differing magnetic strength. For example, the individual magnetic elements may have a substantially cubic shape, circular shape, oval shape, or any shape configured to fit within a targeted blood vessel.

The distal magnetic array 122 may be arranged on or within the catheter body 102 distal to and adjacent the treatment portion 130. Similar to the proximal magnetic array 120, the distal magnetic array 122 may include a plurality of magnetic elements arranged in a longitudinal array along a length of the catheter. Each of the magnetic elements of the plurality may have substantially the same dimensions (e.g., height, width, and depth) and substantially the same magnetic strength. In some embodiments, the plurality of magnetic elements may have a combination of different sized magnets, different shaped magnets, and/or magnets of differing magnetic strength. For example, the individual magnetic elements may have a substantially cubic shape, circular shape, oval shape, or any shape configured to fit within a targeted blood vessel. Each array may have any number of magnetic elements, such as 10 or more, 20 or more, 30 or more, etc.

Similar to the first catheter 100, the second catheter 100′ generally includes a second catheter body 102′ and a second treatment portion 130′. It is noted that the second catheter 100′may include a greater or fewer number of components without departing from the scope of the present disclosure.

The second catheter body 102′ may include a second distal tip 110′ that may be shaped and/or sized to aid in advancement of the second catheter 100′ through a blood vessel. For example, the second distal tip 110′ may be pointed, tapered, and/or atraumatic for advancement through a blood vessel. The second catheter body 102′ may have any cross-sectional shape and any diameter suitable for intravascular use. The second catheter 100′ may include or define one or more passageways (not shown) extending at least partially along or through the second catheter body 102′. For instance, the one or more passageways may extend at least partially longitudinally through the second catheter body 102′ in the direction of the X-axis of the depicted coordinate axes of FIG. 1. The second catheter body 102′ and/or the second treatment portion 130′, may be formed of any material or combination of materials able to be traversed through a vasculature of a body. For example, the second catheter body 102′ and/or the second treatment portion 130′may include, silicone, rubber, etc. The second catheter body 102′ and/or the second treatment portion 130′ may be formed through any material generation process, including but not limited to injection molding or other machining process.

As noted above, the second catheter 100′ may define the second treatment portion 130′that may be configured to be aligned or coapted with the first treatment portion 130 of the first catheter 100 such as when the first catheter 100 and the second catheter 100′ are aligned in adjacent vessels. The second treatment portion 130′ may define a second treatment slot 132′, which may be configured to receive the electrode 142 as it passes through tissue of the adjacent vessels to form a fistula. The second treatment slot 132′ may have a shape (e.g., a concave portion) that corresponds to and is complementary (e.g., inverse, reciprocal) to the electrode 142 to match and conform to the electrode 142 when the first treatment portion 130 and the second treatment portion 130′ are aligned and/or coapted.

The second treatment slot 132′ may be electrically conductive and/or electrically insulative. For example, where electrically conductive, the second treatment slot 132′ may act as an extension of the electrode 142 to aid in ablation or removal of tissue. Where electrically insulative, the second treatment slot 132′ may insulate portions of the body and/or second catheter 100′ from electrical contact by the electrode 142. For example, the second treatment slot 132′ may include a backstop (e.g., a ceramic or other insulative backstop). This may optionally protect other regions of the patient's vasculature from being affected by the electrode 142. Similar to the first treatment slot 132, the second treatment slot 132′ may be adjustable, as described in further detail herein. However, the second treatment slot 132′ may also be non-adjustable, i.e. may be of a fixed length. For example, and in some embodiments, the second treatment slot 132′ may be designed to be the maximum adjustable length of the first treatment slot 132. In embodiments, the first and second treatments slots 132/132′ may have substantially the same length in the un-adjusted positions.

In embodiments, the second catheter 100′ may further include one or more magnetic arrays for alignment or coaptation purposes of the first catheter 100 to the second catheter 100′. The one or more magnetic arrays may be substantially identical to the one or more magnetic arrays for the first catheter 100, with the exception that the polarity may be different to align or coapt the first treatment portion 130 with the second treatment portion 130′ and/or direct the first treatment slot 132 toward the second treatment slot 132′.

While shown in FIG. 1 as being configured to receive the electrode 142, i.e., having a complementary shape, it should also be appreciated that in some variations, the second treatment portion 130′ of the second catheter 100′ may not be configured to receive the electrode 142. In some variations, the second treatment portion 130′ of the second catheter 100′ may include the electrode (not shown), such as described hereinabove, in addition to or instead of the first catheter 100 having the electrode 142. This may optionally increase the contact area for the ablation surface and aid in consistent fistula formation. Accordingly, in some embodiments, the first catheter 100 and the second catheter 100′ may be substantially identical to one another.

It is noted that the first catheter 100 and/or the second catheter 100′ and any accompanying elements may be sized and/or shaped to be advanced through any target blood vessel. For example, in blood vessels having an internal diameter of about 3 mm, it may be desirable to configure any of the catheter elements to be less than about 3 mm in diameter/width. In some embodiments, the first catheter 100 and/or the second catheter 100′ may have any suitable diameter for intravascular use, such as, for example, about 4 French (1.33 mm), about 5.7 French (1.9 mm), about 6.1 French (2.03 mm), about 7 French (2.33 mm), about 8.3 French (2.77 mm), or a value between about 4 French (1.33 mm) and about 9 French (3.0 mm), between about 4 French (1.33 mm) and about 7 French (2.33 mm), between about 4 French (1.33 mm) and about 6 French (2.0 mm), or the like.

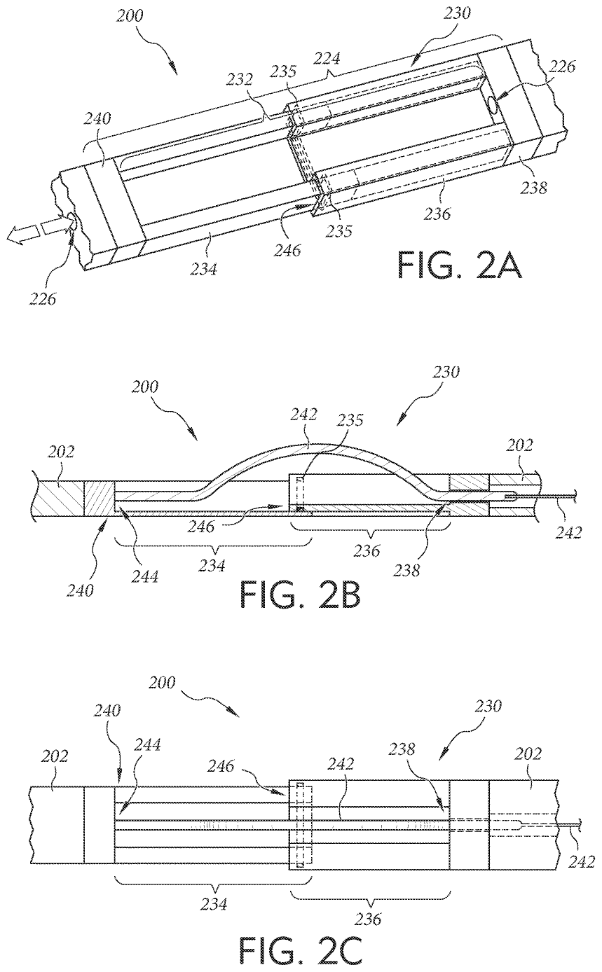

As previously described, the length of the first treatment slot 132 and the second treatment slot 132′ may be adjustable. The lengths may be adjustable according to any one of the embodiments described in further detail hereinbelow and as illustrated in FIGS. 2A through 7C. FIGS. 2A through 7C illustrate different catheter designs for the creation of variable sized fistulas in the systems, as previously described. Each of the catheters described below may also include any of the components previously described in system 10 above. Further, each of the catheters described below may be used as pairs, or as any combination in the system 10 as the first catheter 100 and the second catheter 100′.

Each of the adjustable lengths of the first treatment slot 132 and/or the second treatment slot 132′ described hereinbelow may have lower and upper limits past which adjustment is no longer possible. In other words, the first treatment slot 132 and the second treatment slot 132′ may have a maximum length and a minimum length. The minimum length of the electrode 142 in the longitudinal direction may be bound by the minimum length of the first treatment slot 132. The maximum length of the electrode 142 may be bound by the maximum length of the first treatment slot 132. In the maximum length, the electrode 142 may be completely contained within the first treatment slot 132, such that the electrode 142 is unable to protrude through the first treatment slot 132.

Now referring to FIGS. 2A-2C, illustrated is a ‘nesting’ type adjustable catheter 200. The catheter 200 of FIGS. 2A-2C may be similar or identical in some respects to that of the first catheter 100 and/or the second catheter 100′ of FIG. 1. The catheter 200 may include the treatment portion 230 and the treatment slot 232. The treatment portion 230 may also include or provide an electrode housing 224. The electrode housing 224 may include a first portion 234 and a second portion 236 (shown in isolation in FIG. 2A without the electrode 242), which may also be described herein as an “inner housing” and an “outer housing,” respectively. The first portion 234 and the second portion 236 may together define the treatment slot 232, which may also be referred to herein a “treatment region,” extending between a first end wall 240 of the first portion 234 and a second end wall 238 of the second portion 236. The first portion 234 and second portion 236 may also include or define the one or more passageways 226, as previously described, extending at least partially along or through the catheter body 202, as well as the first and second portions. The electrode 242 may then extend through the one or more passageways 226, such as from first end wall 240 to second end wall 238, or vice versa. The electrode 242 may then be fixed at or within either first end wall 240 or second end wall 238 (such as the opposite end wall to the one or more passageways 226 within which the electrode 242 extends) at an electrode affixing point 244, or it may be fixed at some further distal end of the catheter 200. For example, and as illustrated the electrode 242 may be fixed to the second end wall 238 at the electrode affixing point 244 such as via adhesive, welding, brazing, or other fastening techniques.

Still referring to FIGS. 2A-2C, the catheter 200 of FIGS. 2A-2C may include a treatment slot 232 that may be substantially U-shaped. Accordingly, in embodiments, the first portion 234 and the second portion 236 may also be substantially U-shaped. The first portion 234 may be operable to fit within the second portion 236 through a receiving slot 246 of the second portion 236. The first portion 234 may be operable to slide relative thereto, such as by a tongue-in-groove mechanism, into the second portion 236 as an inner nested component within an outer nesting component. The first end wall 240 of the first portion 234 may define a maximum amount the first portion 234 may slide into the receiving slot 246 of the second portion 236, such that the first end wall 240 may block further sliding of the first portion 234 into the second portion 236. The sliding of the first portion 234 into and out of the second portion 236 may thereby adjust the length of the treatment slot 232.

Referring to FIGS. 2B and 2C, the catheter 200 also includes the electrode 242, as previously described. The electrode 242 may then extend through the one or more passageways 226, from second end wall 238 to first end wall 240. The electrode 242 may be fixed at or within first end wall 240 at an electrode affixing point 244, or it may be fixed at some further distal end of the catheter 200. In embodiments, sliding of the first portion 234 out of the second portion 236 through the receiving slot 246 may operate to increase the length of the treatment slot 232. The electrode 242 may slide through the one or more passageways 226 of the second end wall 238, thereby providing a larger exposed portion of the electrode to protrude from the treatment slot 232. In a similar manner, sliding of the first portion 234 into the second portion 236 through the receiving slot 246 may operate to reduce the length of the treatment slot 232. The electrode 242 may be slid proximally through the one or more passageways 226 of the second end wall 238 such that a shorter exposed portion of the electrode 242 protrudes from the treatment slot 232. Thereby, the size of a fistula may be adjusted to be larger or smaller depending on the desired resultant fistula size.

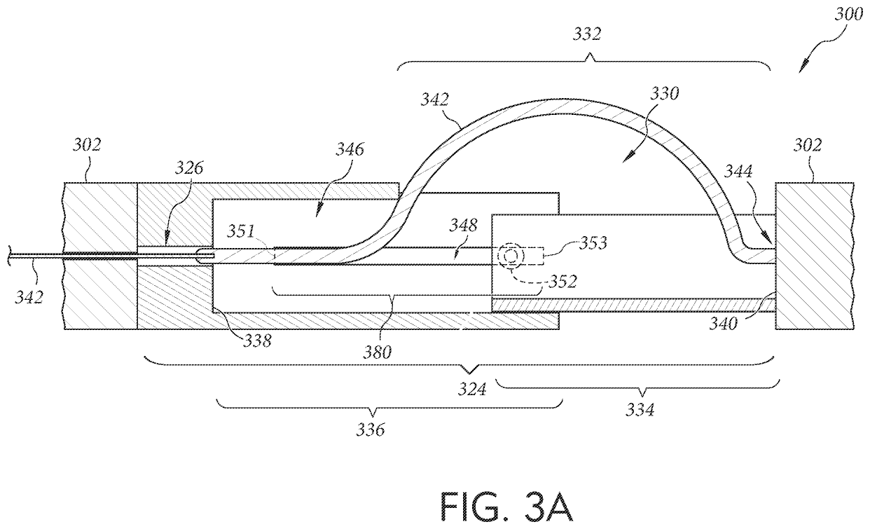

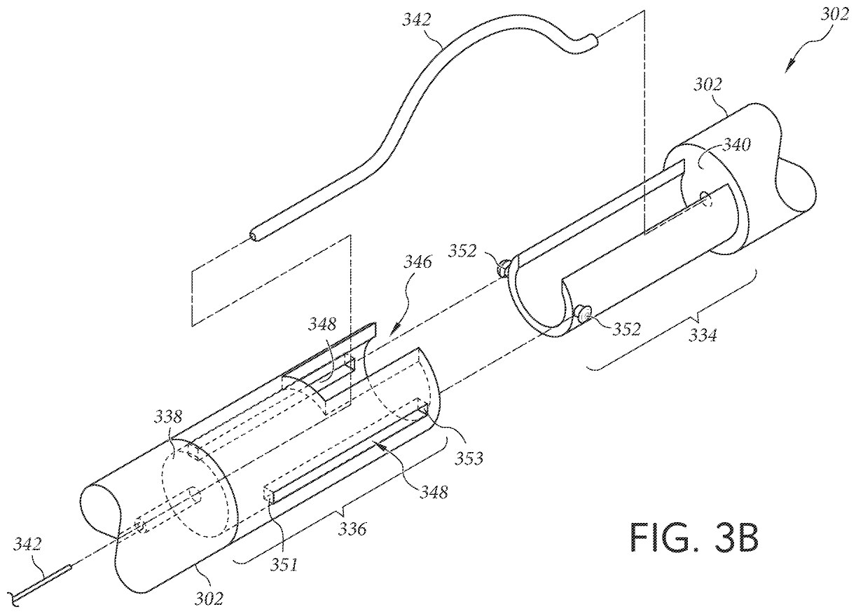

Now referring to FIGS. 3A and 3B, illustrated is a dual-housing, slidably adjusted catheter. The catheter 300 of FIGS. 3A and 3B may be similar or identical in some respects to that of the catheter 200 of FIGS. 2A-2C. The catheter 300 may include the treatment portion 330, which may include an electrode housing 324 that may in turn include the first portion 334 and the second portion 336, which may also be described as the inner housing 334 and the outer housing 336, respectively, as hereinbefore described. Similar to FIGS. 2A and 2B, the outer housing 336 of FIGS. 3A and 3B may define a receiving slot 346 positioned within the treatment portion 330 and within the outer housing 336, the receiving slot 346 is sized to receive the inner housing 334. The receiving slot 346 may then further define one or more slots 348 extending from the receiving slot 346 through the outer housing 336. Though the receiving slot 346 are illustrated as extending to the exterior of the outer housing 336, they may instead only extend a portion of the distance to the exterior of the outer housing 336. The one or more slots 348 may also be bound within the outer housing, such that the one or more slots 348 are defined by slot proximal end 351 and slot distal end 353.

Still referring to FIGS. 3A and 3B, one or more pins 352 may protrude from an exterior of the inner housing 334, such that when the inner housing 334 is positioned within the receiving slot 346, the one or more pins 352 may be positioned within the one or more slots 348. Thereby, the one or more slots 348 and the one or more pins 352 may together make up a sliding limit assembly 380. The sliding limit assembly 380 may then be bounded by the slot proximal end 351 and the slot distal end 353. The inner housing 334 may be slidably positioned within the receiving slot 346, such that the one or more pins 352 are positioned within the one or more slots 348. Accordingly, sliding of the inner housing 334 relative to the outer housing 336 is limited by the one or more pins 352 moving along the one or more slots 348 between the slot distal end 353 and the slot proximal end 351. Accordingly, the longitudinal length of the treatment slot 332 and the electrode 342 is adjustable in a manner similar to that discussed with respect to embodiments above.

Although not shown, the one or more pins 352 may be removably coupled to the inner housing 334 through any means known in the art. Thereby, the inner housing 334, without the one or more pins 352, may be initially positioned within the receiving slot 346. Subsequently, the one or more pins 352 may be coupled to the outer housing to make up the sliding limit assembly 380. The one or more pins 352 may also be positioned on an exterior of the inner housing 334 proximal to the first end wall 340, or, as illustrated in FIG. 3B, distal to the first end wall 340.

Still referring to FIGS. 3A and 3B, the electrode 342 may extend through the one or more passageways 326, such as from the first end wall 340 to the second end wall 338. The electrode 342 may be fixed at or within the first end wall 340 at an electrode affixing point 344, or it may be fixed at some further distal end of the catheter 300. Thereby, adjustment of the inner housing 334 along the one or more slots 348 through the one or more pins 352 towards the first end wall 340 and/or the slot distal end 353 may operate to increase the length of the treatment slot 332 and allow a greater longitudinal length of the electrode 342 to protrude from the catheter 300, as described above. In a similar manner, adjustment of the inner housing 334 along the one or more slots 348 through the one or more pins 352 towards the second end wall 338 and/or the slot proximal end 351 may operate to decrease the length of the treatment slot 332 and allow a shorter longitudinal length of the electrode 342 to protrude. Accordingly, the length of exposed electrode for fistula creation may be adjusted depending on the desired fistula size.

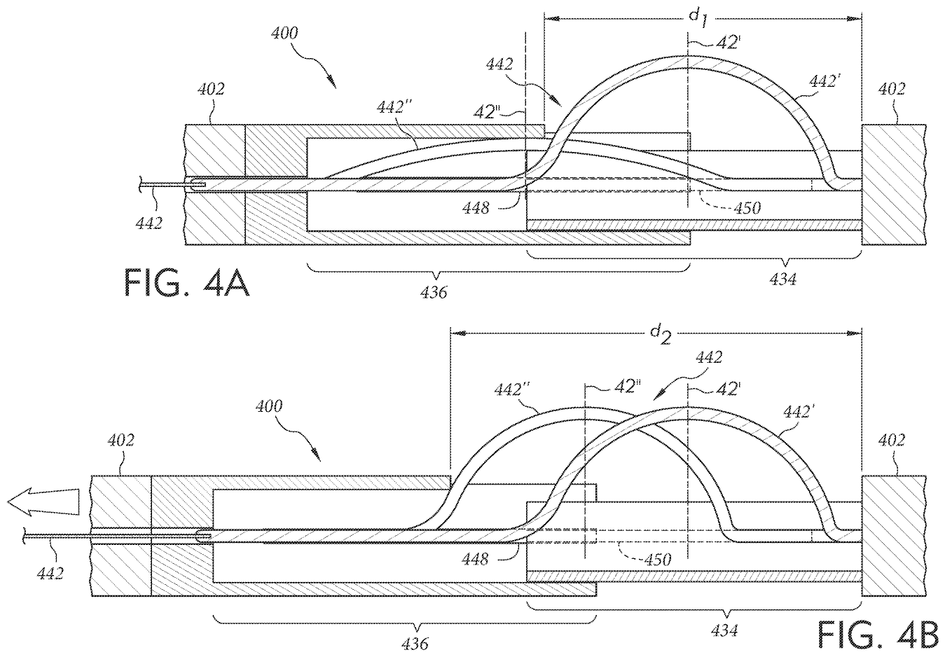

Now referring to FIGS. 4A and 4B, illustrated is a catheter 400 with dual electrodes. The catheter 400 of FIGS. 4A and 4B may be similar or identical in some respects to that of the catheters of FIGS. 2A-3B. Similar to FIGS. 3A-3B, the catheter 400 of FIGS. 4A and 4B may include the inner housing 434 and the outer housing 436. The catheter 400 of FIGS. 4A and 4B may also include the one or more slots 448, as well as one or more grooves 450, the one or more slots sized to fit the one or more grooves 450. In embodiments, the inner housing 434 and the outer housing 436 may have any of the structures as described above. Accordingly, the outer housing 436 may be slidable along the inner housing 434, or vice versa, to adjust the length of the treatment slot 432.

In the present embodiment, the catheter 400 of FIGS. 4A and 4B, instead of a single electrode as shown in embodiments above, includes a first electrode 442′ and a second electrode 442″. The second electrode 442″ may be longitudinally offset from the first electrode 442′, e.g., an apex 42′ of the first electrode 442′ may be longitudinally offset from an apex 42″ of the second electrode 442″. For example, the outer housing 436 may be adjusted to a first length di of the treatment slot 432, which may expose only the first electrode 442′ and allow only the first electrode 442′ to protrude through the treatment slot 432. The outer housing 436 may instead be adjusted to a second length de of the treatment slot 432, wherein d₂ is greater than d₁ to expose the second electrode 442″ and allow the second electrode 442″ to protrude through the treatment slot 432. The exposure of the second electrode 442″ may operate to increase the contact area of the electrode 442. Accordingly, the size of a fistula may be adjusted by exposing or covering additional electrodes.

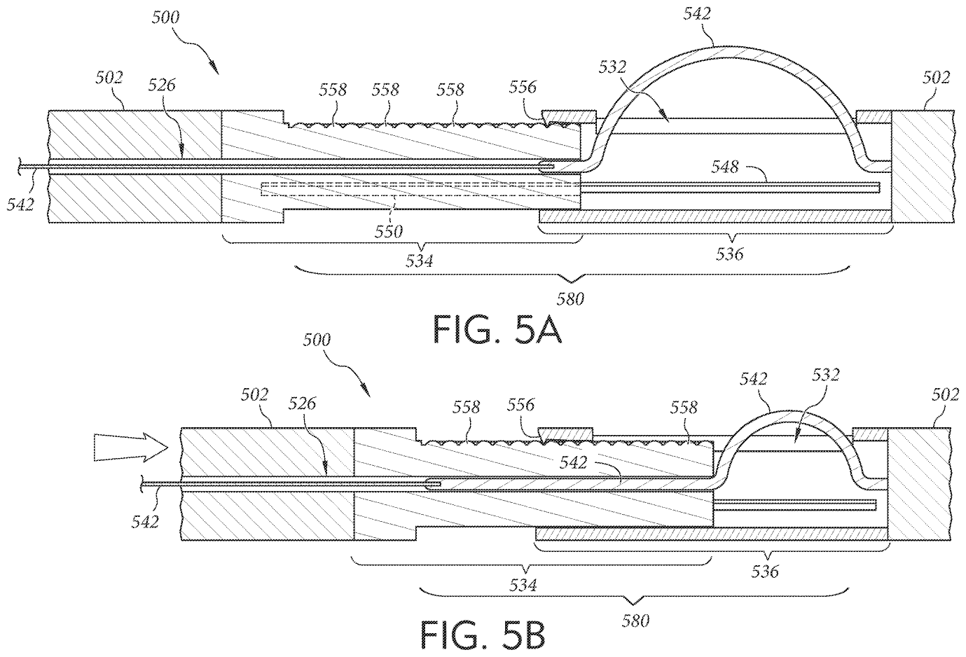

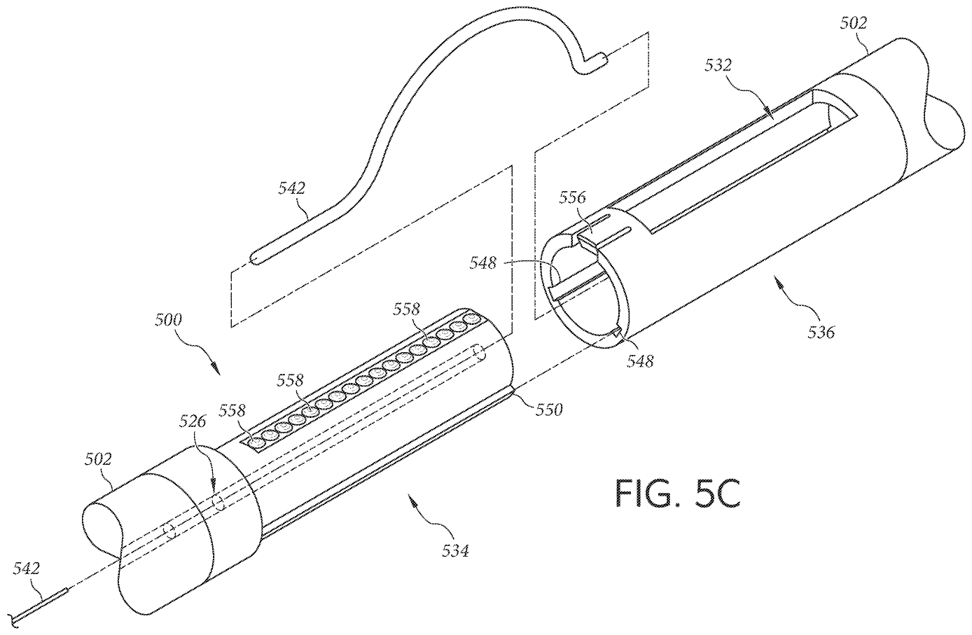

Now referring to FIGS. 5A-5C, illustrated is a dual-housing slidably adjusted catheter 500. The catheter 500 may include a sliding limit assembly 580 including a plurality of ridges 558 and one or more protrusions 556. The catheter 500 of FIGS. 5A-5C may be similar or identical in some respects to that of the catheters of FIGS. 2A-4B. Similar to the previous catheters, the catheter 500 of FIGS. 5A-5C may include the outer housing 536 and the inner housing 534. In embodiments, the catheter 500 of FIGS. 5A-5C may also include the sliding limit assembly features described above, e.g., the catheter 500 of FIGS. 4A and 4B may include the one or more slots, one or more grooves, and/or one or more pins. In the depicted embodiment, the sliding limit assembly 580 includes a plurality of ridges 558 formed on one of the inner housing 534 or the outer housing 536 (not shown), as well as one or more protrusions 556 formed on the other of the outer housing 536 and the inner housing 534 (not shown). The plurality of ridges 558 may be proportionally spaced along the length of one of the inner housing 534 or the outer housing 536. The inner housing 534 may then be slidably adjusted to ‘catch’ on one of the plurality of ridges 558 with the one or more protrusions 556 to secure the inner housing 534 in a position relative to the outer housing 536. The plurality of ridges 558 may allow the inner housing 534 to be secured at the one or more protrusions 556 to adjust the length of the treatment slot 532. Thereby, the plurality of the ridges 558 and the one or more protrusions 556 may together make up the sliding limit assembly 580. The one or more protrusions 556 and the plurality of ridges 558 may also be circumferentially spaced around the inner housing 534 and the outer housing 536, such that two or more protrusions 556 may ‘catch’ on two or more of the plurality of ridges 558. It is contemplated that additional ‘catch’ points may add strength to the connection point of the inner housing 534 and outer housing 536, and thereby reduce the chance of the inner housing 534 and/or outer housing 536 changing positions when used. The catheter 500 may also optionally include one or more grooves 550 as well as one or more slots 548, to orient the inner housing 534 within the outer housing 536, as well as the plurality of ridges 558 in track with the one or more protrusions.

Still referring to FIGS. 5A and 5B, the electrode 542 may extend through the one or more passageways 526 which may be formed in the first end wall 540 or second end wall 538. The electrode 542 may be fixed at or within the second end wall 538 at an electrode affixing point 544, or it may be fixed at some further distal end of the catheter 500. Thereby, adjustment of the inner housing 534 in the distal direction, i.e. further into the outer housing 536 and the second end wall 538, may operate to reduce the length of the treatment slot 532 and thereby reduce the longitudinal length the electrode 542 is exposed from the catheter 500. In a similar manner, adjustment of the inner housing 534 in a proximal direction, i.e. away from the second end wall 538 and out of the outer housing 536, may operate to increase the length of the treatment slot 532 and thereby increase a longitudinal length the electrode 542 is exposed from the catheter 500. Accordingly, the size of the resulting fistula may be selected based on positioning of the inner housing 534 and the outer housing 536 relative to one another. The inner housing 534 and the outer housing 536 may be held in place via interaction between the plurality of ridges 558 and the one or more protrusions 556.

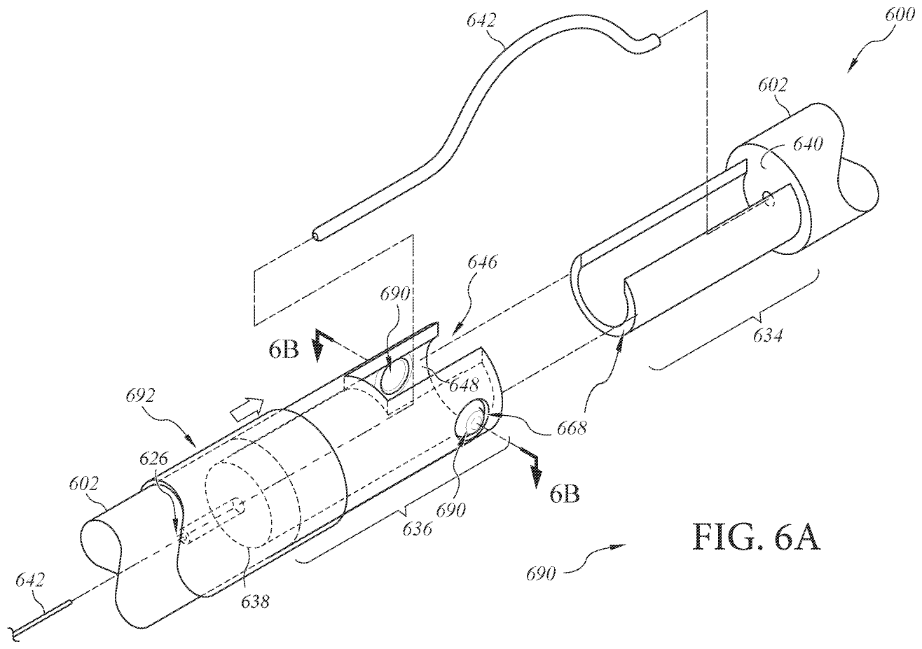

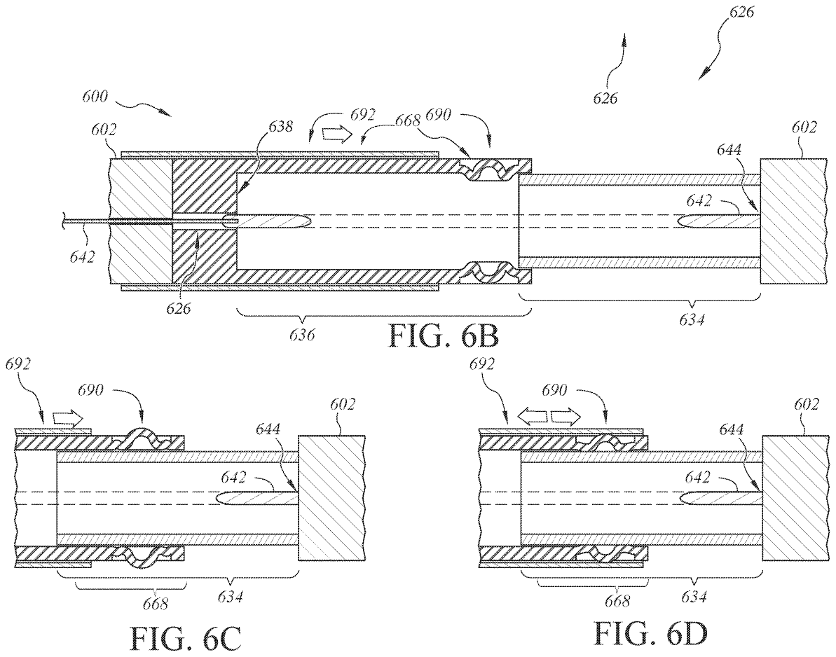

Referring to FIGS. 6A-6D, another embodiment of a dual-housing catheter 600 is depicted. The catheter 600 of FIGS. 6A-6D may be similar or identical in some respects to that of the catheters of FIGS. 2A-5C. In particular, as with the previous described catheters, the catheter 600 of FIGS. 6A-6D may include an outer housing 636, an inner housing 634, and an interlocking mechanism 668. The interlocking mechanism 668 may include a protruding section 690 and a sliding sleeve 692. As shown in FIG. 6B, the protruding section 690 may be bumps, nubs, or the like, which may protrude from the exterior and interior of the outer housing 636, and which may themselves be a part of the outer housing 636. Accordingly, the protruding section 690 may also include or be formed of elastic material. The elasticity of the protruding section 690 may allow the protruding sections 690 to be deformed radially inward under force (e.g., compression) so as to engage the inner housing 634, as described in greater detail below. Upon release of the force, the elasticity of the protruding section 690 may allow the protruding section to return to its pre-compressed state. The protruding section 690 may include a single projection, several projections (as shown), or in some embodiments a radially extending projection that encompasses the entire perimeter of the outer housing 636.

As noted above, a sliding sleeve 692 may be included and operable to apply force (e.g., compressive force) to the protruding section 690. The sliding sleeve 692 may be positioned on the exterior of the outer housing 636 and operable to slide over the protruding section 690. During assembly, as shown in FIG. 6C, insertion of the inner housing 634 past the protruding section 690 may operate to cause the protruding section 690 to contact an exterior of the inner housing 634 and may or may not cause the protruding section 690 to bulge further outward. As shown in FIG. 6D, the sliding of the sliding sleeve 692 over the bulging protruding section 690 may operate to compress the protruding section 690 over the inner housing 634, thereby providing a friction lock between the inner housing 636 and outer housing 634.

Still referring to FIGS. 6A-6D, and similar to the previous catheters 100-500, the electrode 642 may extend through the one or more passageways 626 which may be formed in the first end wall 640 or the second end wall 638. As shown in FIGS. 6A-6D, the electrode 642 may be fixed at or within the first end wall 640 of the inner housing 634 at an electrode affixing point 644, or it may be fixed at some further distal end of the catheter 600. Accordingly, similar to the catheter 300 of FIGS. 3A-3B, adjustment of the inner housing 634 in the proximal direction, i.e. into the outer housing 636 and towards the second end wall 638, may operate to reduce the length of the treatment slot 632 and thereby reduce the longitudinal length the electrode 642 is exposed from the catheter 600. In a similar manner, adjustment of the inner housing 634 in a distal direction, i.e. out of the outer housing 636 and in the direction of the first end wall 640, may operate to increase the length of the treatment slot 632 and thereby allow a lesser longitudinal length of the electrode 642 to protrude from the catheter 600. Accordingly, the size of the resulting fistula may be selected based on positioning of the inner housing 634 and the outer housing 636 relative to one another. As previously mentioned, the inner housing 634 and the outer housing 636 may then be held in place via sliding of the sliding sleeve 692 over the protruding section 690. However, in some embodiments the catheter 600 may not include the sliding sleeve 692, with the contact of the protruding section 690 with the inner housing 634 providing sufficient resistance to hold the inner and outer housings 634, 636 in place.

For example, in embodiments the catheter body 602, the inner housing 634, and/or the outer housing 636, may include or be formed of an elastic material, such that the catheter body 602, the inner housing 634, and/or the outer housing 636, may be radially stretchable or radially compressible. The elastic and/or deformable material may include the materials for the catheter body previously discussed, such as silicone, rubber, etc. Particularly, the outer housing 636 may include elastic material with requisite elasticity such that the outer housing 636 is at least partially stretched and/or deformed when placed around the inner housings 636. Accordingly, the outer housing 636 may grip the inner housing 636. However, in other embodiments, the inner housing 634 may be formed of a deformable material which is radially compressible. In such embodiment, the inner housing 634 may be compressed by the outer housing 634 such that a frictional engagement is formed between the inner housing 634 and the outer housing 636. In such embodiments, it is contemplated that there may not be any protruding section. For example, the interlocking mechanism may be provided via the radially expandable or compressible inner or outer housing 634, 636.

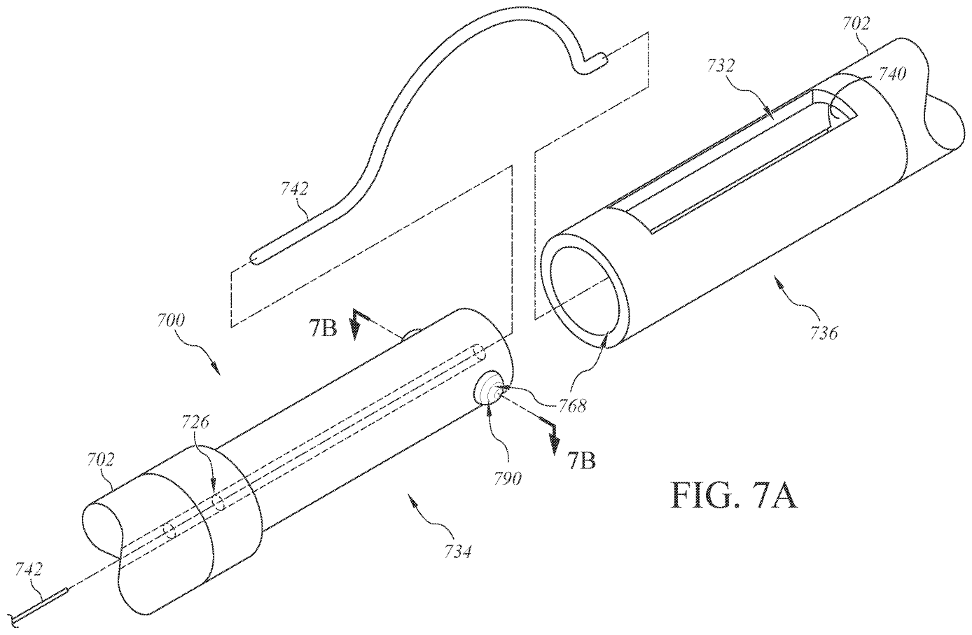

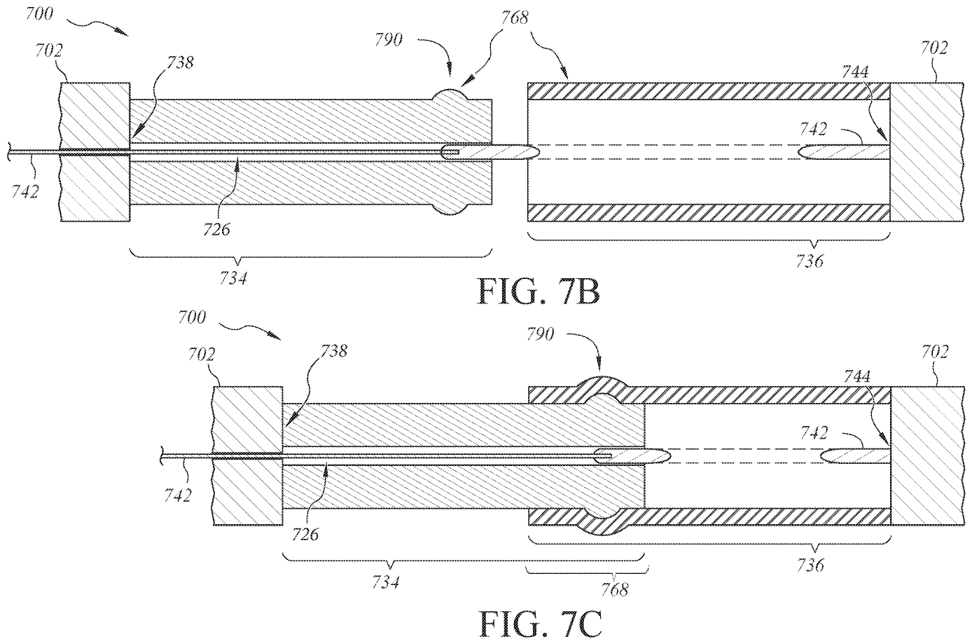

Now referring to FIGS. 7A-7C, another embodiment of a dual-housing catheter 700 is depicted. The catheter 700 of FIGS. 7A-7C may be similar or identical in some respects to that of the catheters of FIGS. 2A-6D. Similar to the previous catheters, the catheter 700 of FIGS. 7A-7C may include the outer housing 736, the inner housing 734, and an interlocking mechanism 768. However, in the present embodiment, the interlocking mechanism 768 is shown as including a protruding section 790 formed on the inner housing 734, For example, and as shown in FIG. 7B, the protruding section 790 may be bumps, nubs, or the like, which may protrude from the exterior of the inner housing 734, and which may themselves be a part of the inner housing 734. The protruding section 790 may include a single projection, several projections (as shown), or in some embodiments a radially extending projection that encompasses the entire perimeter of the inner housing 734. For example, the protruding section 790 may be a tapered mandrel or wedge shape (not shown), such that insertion of the inner housing 734 into the elastic outer housing 736 may become increasingly difficult the further the inner housing 734 is inserted into the elastic outer housing.

Similar to the above description, the outer housing 736 may include or be formed of elastic material. As shown in FIG. 7C, the elasticity of the outer housing 736 may allow the outer housing 736 to be deformed radially outward under force (e.g. tension) so as to engage the inner housing 734 by a frictional fit. That is, inserting the inner housing 734 into the outer housing 736 may stretch or radially expend the outer housing 736 thereby allowing the outer housing 736 to grip the inner housing 734 such as via frictional engagement. As shown in FIG. 7B, upon release of the force (e.g. extraction of the inner housing 734 from the outer housing 736), the elasticity of the outer housing 736 may allow the outer housing to return to its pre-tensioned state. In other embodiments, it is contemplated that the inner housing 734 may instead be radially compressible such that insert of the inner housing 734 into the outer housing 736 compresses the inner housing 734 thereby allowing the inner housing 734 to form a friction grip with the outer housing 736. In some embodiments, and as noted above, the inner or outer housings 734/736 may not include the protrusions and may instead rely on the elastic and/or compressible material which may form the inner or outer housings 734/736.

Still referring to FIGS. 7A-7C, and similar to the previous catheters 100-600, the electrode 742 may extend through the one or more passageways 726 which may be formed in the first end wall 740 or second end wall 738. The electrode 742 may be fixed at or within the second end wall 738 at an electrode affixing point 744, or it may be fixed at some further distal end of the catheter 700. Thereby, adjustment of the inner housing 734 in the distal direction, i.e. into the outer housing 736 and towards the second end wall 738, may operate to reduce the length of the treatment slot 732 and thereby decrease the longitudinal length the electrode 742 is exposed from the catheter 700. In a similar manner, adjustment of the inner housing 734 in a proximal direction, i.e. out of the outer housing 736 in in the direction of the first end wall 740, may operate to increase the length of the treatment slot 732 and thereby increase the longitudinal length the electrode 742 is exposed from the catheter 700. Accordingly, the size of the resulting fistula may be selected based on positioning of the inner housing 734 and the outer housing 736 relative to one another. As previously mentioned, the inner housing 734 and the outer housing 736 may then be held in place via the frictional fit between the outer housing 736 and the inner housing 734.

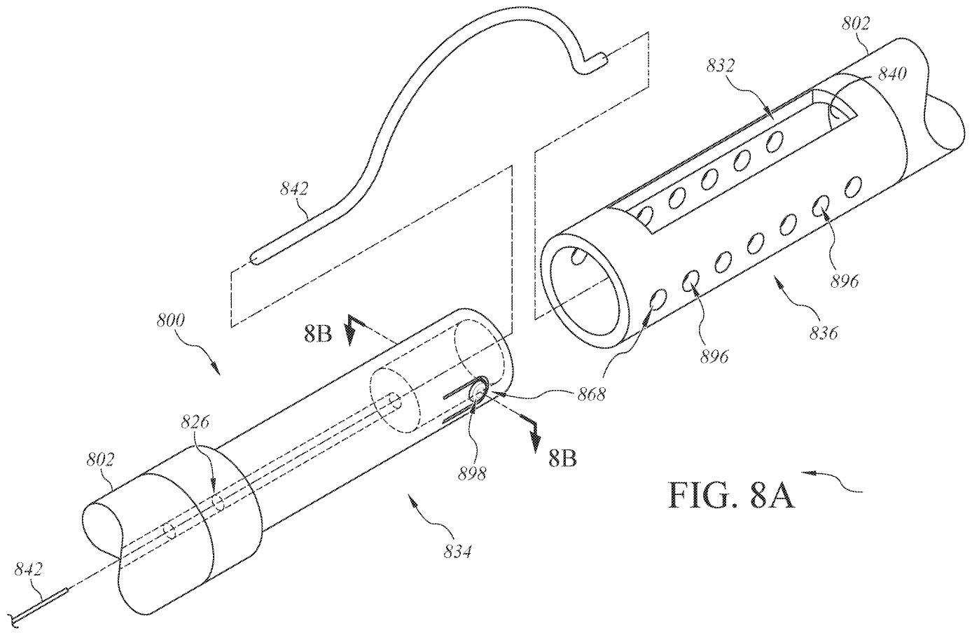

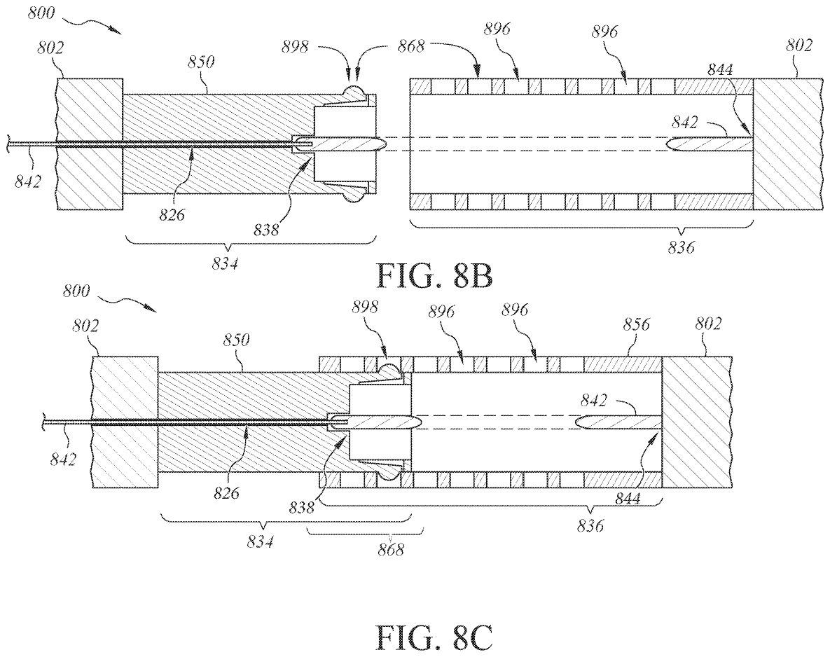

Now referring to FIGS. 8A-8C, illustrated is another embodiment of a dual-housing catheter 800. The catheter 800 of FIGS. 8A-8C may be similar or identical in some respects to that of the catheters of FIGS. 2A-7C. Similar to the previous catheters, the catheter 800 of FIGS. 8A-8B may include the outer housing 836, the inner housing 834, and an interlocking mechanism 868. As shown in FIGS. 8A-8B, the interlocking mechanism 868 may include a plurality of bores 896 extending through the outer housing 836. The interlocking mechanism 868 may further include one or more depressible buttons 898 that may be coupled to and protrude from the exterior of the inner housing 834. The one or more depressible buttons 898 may be configured to be depressible through a spring-loading mechanism, a snap button arrangement, or combinations thereof, although other methods are contemplated. As shown in FIG. 8C, the depressibility of the one or more depressible buttons 898 may allow the depressible buttons 898 to be compressed radially inward under force (e.g. compression) so as to become depressed for insertion into the outer housing 836. As shown in FIG. 8B, upon release of the force, the one or more depressible buttons 898 may return to their pre-compressed state, such as due to the aforementioned spring-loading mechanism or snap button arrangement.

In embodiments, the plurality of bores 896 may be sized to receive the one or more depressible buttons 898. Accordingly, depression of the one or more depressible buttons 898 may allow insertion of the inner housing 834 into the outer housing 836. As shown in FIG. 8C, upon passing the one or more depressible buttons 898 passed the one or more of the plurality of bores 896, the one or more depressible buttons 898 may return spring outward toward their pre-compressed state and into the one or more of the plurality of bores 896. The extension of the one or more depressible buttons 898 within one or more of the plurality of bores 896 may thereby engage the outer housing 836 and hold it in place relative to the inner housing 834. Depression of the one or more depressible buttons 898 again may allow the inner housing 834 to be inserted farther into the outer housing 836, thus allowing adjustment of the treatment slot 832 length of the catheter 800. In such manner, the plurality of bores 896 and the one or more depressible buttons 898 may function similar to a snap button such as for telescoping poles. Similar to FIGS. 5A-5C, the catheter 800 may also optionally include one or more grooves as well as one or more slots, to orient the inner housing 834 within the outer housing 836, as well as the one or more depressible buttons 898 in track with the plurality of bores 896.

Still referring to FIGS. 8A-8C, and similar to the previous catheter 100-700, the electrode 842 may extend through the one or more passageways 826 which may be formed in the first end wall 840 or the second end wall 838. The electrode 842 may be fixed at or within the second end wall 838 at an electrode affixing point 844, or it may be fixed at some further distal end of the catheter 800. Thereby, adjustment of the inner housing 834 in the distal direction, i.e. into the outer housing 836 and towards the second end wall 838, may operate to reduce the length of the treatment slot 832 and thereby decrease the longitudinal length the electrode 842 is exposed from the catheter 800. In a similar manner, adjustment of the inner housing 834 in a proximal direction, i.e. out of the outer housing 836 in in the direction of the first end wall 840, may operate to increase the length of the treatment slot 832 and thereby increase the longitudinal length the electrode 842 is exposed from the catheter 800. Accordingly, the size of the resulting fistula may be selected based on positioning of the inner housing 834 and the outer housing 836 relative to one another. As previously mentioned, the inner housing 834 and the outer housing 836 may then be held in place via the frictional fit between the outer housing 836 and the inner housing 834, through the plurality of bores 896 and the one or more depressible buttons 898.

Referring back to FIGS. 2A-8C, in embodiments, the catheters may also include one or more additional features. For example, in embodiments, and as shown in FIGS. 2A-2B, the first portion 234, the second portion 236, or both may include a coating or lining element 235, including but not limited to rubbers, synthetic rubbers, thermoplastics, polymer films, or combinations thereof. The coating or lining element may be in the form of a gasket or O-ring. Alternatively or additionally, the coating or lining element 235 may be formed by sand blasting or etching the first portion 234, the second portion 236, or both. As shown in FIGS. 2A-2B, and in embodiments, the coating or lining element 235 may operate to allow for holding of a chosen treatment slot length by a friction fit between the first portion 234 (the inner housing) and the second portion 236 (the outer housing). For example, and in some embodiments, the sand blasting or etching may form a coating or lining element 235 that may increase the coefficient of friction between the first portion 234 and the second portion 236. Similarly, the addition of the coating or lining element 235, in the form of the gasket or O-ring to the first portion 234 (the inner housing) and/or the second portion 236 (the outer housing), may operate to assist in mating the two elements as well as seal the same from intrusion by bodily fluids. In other words, the gasket or o-ring may be configured to at least partially resist sliding of the first portion relative to the second portion. In some embodiments, the first portion 234 (the inner housing) may be locked into a chosen slideable position within the second portion 236 (the outer housing) by the use of an interference feature, such as compressible buttons into a latch (not shown). Alternatively, in the event a reduction in friction between the first portion 234 and the second portion 236 is desired, the coating or lining element 235 may be formed by electropolishing or coating with a metallic alloy, such as titanium nitride. The electropolishing and/or metallic alloy coating may reduce that coefficient of friction between the first portion 234 and the second portion 236, affecting the ease by which the first portion 234 may slide on the second portion 236, or vice versa.

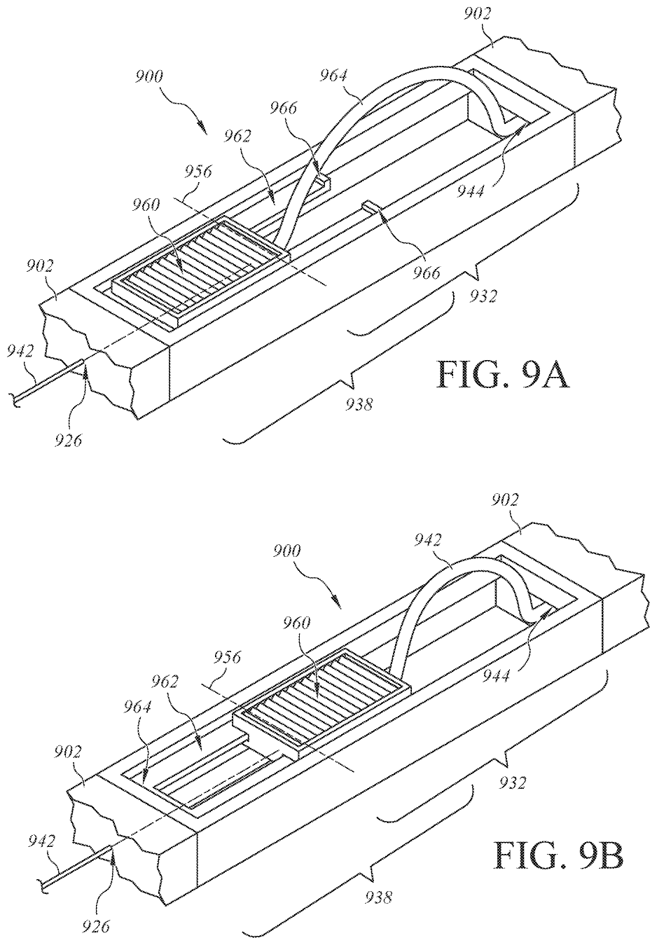

Now referring to FIGS. 9A and 9B, illustrated is the catheter 900 in a single housing with a sliding cover 960 for adjusting the treatment slot 932 length before (FIG. 9A) and after moving (FIG. 9B) the sliding cover 960. The catheter 900 of FIGS. 9A and 9B may be similar or identical in some respects to that of the catheter 900 of FIGS. 2A-5C. In embodiments, the treatment portion 930 of FIGS. 9A and 9B may include the sliding limit assembly 980. The sliding limit assembly 680 may include a sliding cover 960 and a track 962. The sliding cover 960 may be operable to slide along the track 962 to either expose or cover up the treatment slot 632. The track 962 may include a track proximal stop 964 and a track distal stop 966. The track proximal stop 964 and track distal stop 966 of the track 962 may operate to define minimum and maximum distances, respectively, the sliding cover 960 may slidably adjust to cover the treatment slot 632.

Still referring to FIGS. 9A and 9B, and similar to the previously discussed catheters, the electrode 942 may extend through the one or more passageways 926, such as from one end of the treatment portion 930 to the other. The electrode 942 may be fixed at the electrode affixing point 944, or it may be fixed at some further distal end of the catheter 900. Thereby, adjustment of the sliding cover 960 in a proximal direction to the track proximal stop 964 may operate to increase the length of the treatment slot 632 and allow a longer longitudinal length of the electrode 942 to protrude from the catheter 900. In a similar manner, adjustment of the adjustment of the sliding cover 960 in a distal direction to the track distal stop 966 may operate to decrease the length of the treatment slot 932 and allow a smaller longitudinal length of the electrode 942 to protrude from the catheter 900 than when the sliding cover 960 is positioned more proximally. Accordingly, the size of the resulting fistula may be selected based on positioning of sliding cover 960.

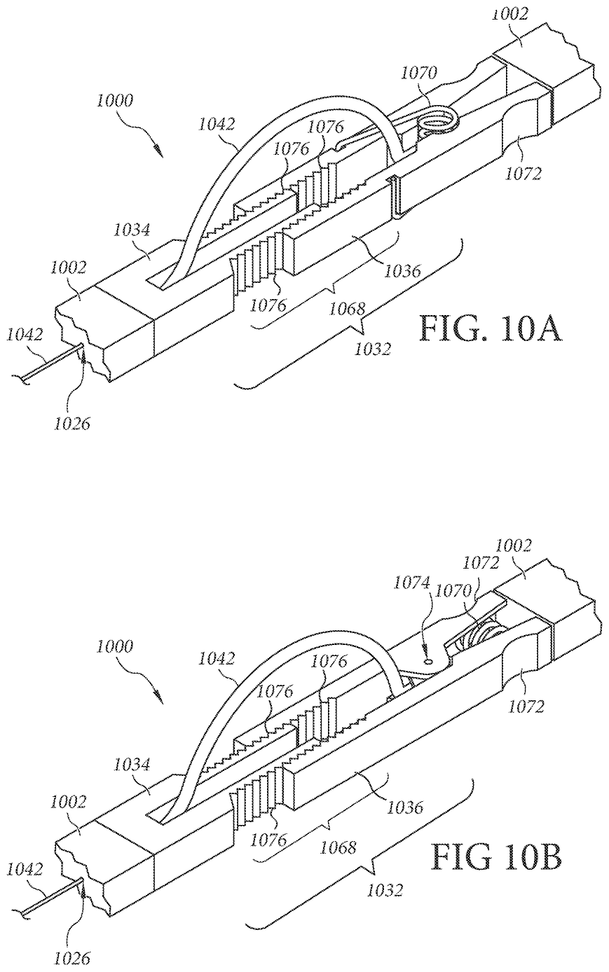

Now referring to FIGS. 10A-10B illustrated is a catheter 1000 with another interlocking mechanism 1068 for the first portion 1034 and the second portion 1036, which may be potentially adjustable by the activation of a spring 1070 through the use of a compression element 1072. The catheter 1000 of FIGS. 10A-10B may be similar or identical in some respects to that of the catheters of FIGS. 2A-9B. As illustrated in FIGS. 10A and 10B, the catheter 1000 may include a treatment portion 730 that may include the first portion 1034 and the second portion 1036. The second portion 1036 may be adjustably coupled to the first portion 1034 via the interlocking mechanism 1068. As illustrated in FIG. 10A, the interlocking mechanism 1068 may include a plurality of interlocking teeth 1076 formed on both the first portion 1034 and the second portion 1036, although other interlocking mechanisms are contemplated.

In embodiments, the second portion 1036 may further include a spring 1070 operable to free the first portion 1034 from the interlocking mechanism 1068 when the compression element 1072 is activated. As illustrated in FIG. 10A, the spring 1070 may be formed as a torsion spring 1070, similar to a laundry pin. Compression of the compression element 1072 may cause the torsion spring 1070 to wind tighter while allowing the region of the second portion 1036 with the interlocking teeth to open and free the first portion 1034 from the interlocking mechanism 1068. The first portion 1034 may then be adjusted to mate with the second portion 1036 at a new location with the interlocking teeth. In some embodiments, as illustrated in FIG. 10B, instead of the torsion spring 1070, the compression element 1072 may be formed as a linear spring 1070 and hinge 1074. Similar to the torsion spring 1070, compression of the linear spring 1070 through the compression element 1072 may operate to open the region of the second portion with the interlocking teeth and free the first portion 1034 from the interlocking mechanism 1068. In either embodiment, once compression on the compression element 1072 is released, the plurality of teeth of the second portion may close toward one another to clasp the first portion 1034.

Still referring to FIGS. 10A and 10B, and similar to the previously discussed catheters, the electrode 1042 may extend through the one or more passageways 1026, such as from one end of the treatment portion 730 to the other. The electrode 1042 may be fixed at or within the second portion 1036, or it may be fixed at some further distal end of the catheter 1000. Thereby, adjustment of the first portion 1034 to a different portion of the interlocking mechanism may operate to change the length of the treatment slot 1032 and thereby the longitudinal length of the electrode 1042 extending through the treatment slot 1032. Accordingly, the resulting size of a fistula may be adjusted by adjusting the position of the first portion 1034 and the second portion 1036 relative to one another.

Now referring to FIG. 10, illustrated is a handle 1178 for use with any of the adjustable catheters herein. The handle 1178 may include one or more user input devices to cause adjustment of the inner housing 834, the outer housing 836, the push/pull rod 1182, the electrode 1142, or combinations thereof, thereby adjusting the length of the treatment slot 832 and/or the longitudinal length of the electrode 1142 extending through the treatment slot 832. In this manner, the one or more user input devices may also be regarded as part of a sliding limit assembly 1180. For example, and as illustrated in FIG. 10, the user input device (sliding limit assembly 1180) may include a plurality of catches 1184, as well as a slider 1186. The slider may be coupled to the push/pull rod 1182, (which may contain the electrode 1142), or the electrode 1142 itself. The push/pull rod 1182 may also be coupled to the first portion or the second portion (i.e. the first and second portions depicted variously in FIGS. 2A-10B, not shown in FIG. 10). In this manner, adjustment of the slider 1186 along the plurality of catches 1184 may operate to translate the push/pull rod 1182 and thereby the first portion or second portion in a controlled manner. In some embodiments, there may be separate adjustment user input devices and/or push/pull rod 1182 for the electrode 1142, the inner housing 834 and/or the outer housing 836. In embodiments, the combination of the push/pull rod 1182, plurality of catches 1184, and slider 1186 may function similar to a linear retractable eraser. In this manner, the length of the treatment portion 130 or 130′ may optionally be adjusted while the catheter 100 or 100′ is located inside a patient's vasculature. Alternatively, in a similar manner, the longitudinal length of the electrode 142 may be adjusted while the catheter 100 or 100′ is located inside a patient's vasculature.

For example, with reference to FIGS. 2A to 2C, translating the slider may operate to translate the linear motion of the slider into movement of the push/pull rod, which in turn adjusts a position of the first portion 234 relative to the second portion 236, and may further adjust a longitudinal length of the electrode 242, and thereby the degree of protrusion from the treatment slot 232. As will be appreciated the handle 1178 may be used in various embodiments described herein.

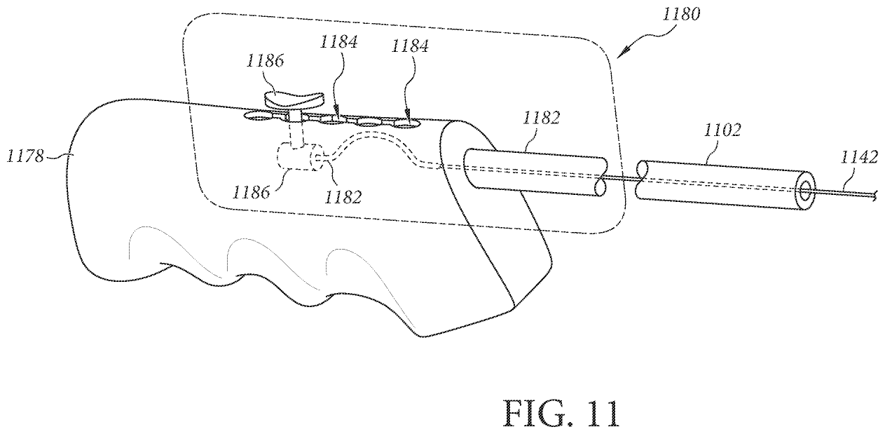

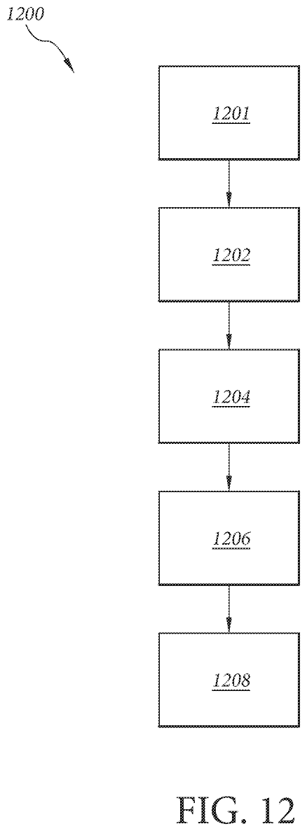

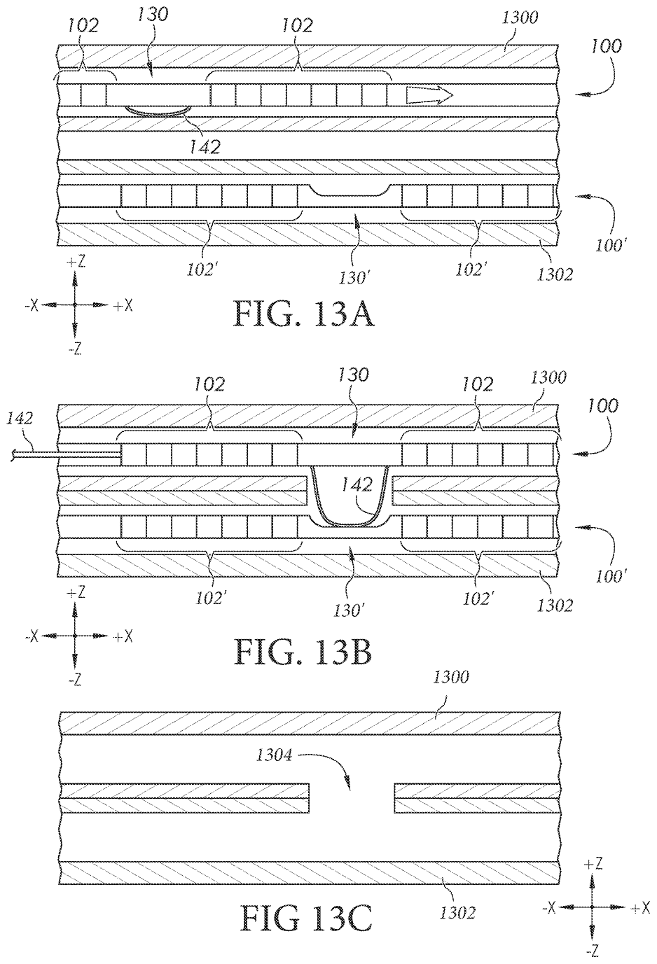

Referring now to FIG. 11, a flow chart illustrating a method 1200 of forming a fistula is generally illustrated. It is noted that the method 1200 may include a greater or fewer number of steps, taken in any order, without departing from the scope of the present disclosure. It is noted that the method 1200 illustrated in FIG. 11 may be best understood when reviewed in conjunction with FIGS. 13A-13C, which generally illustrate alignment and treatment of a blood vessel using the first catheter 100 and the second catheter 100′ as described herein.

Still referring to FIG. 11, at block 1201 the method 1200 includes adjusting the treatment slot 132 length of the first catheter 100, the second catheter 100′, or both, using one of the systems 10 herein, to define an exposed electrode length, and thereby a planned fistula size. Block 1201 may alternatively include adjusting the longitudinal length of the electrode 142 to adjust a distance the electrode 142 protrudes from the length of the first catheter 100, using one of the systems 10 herein, such as through the handle 1178′s plurality of catches 1184 and the slider 1186, through the push/pull rod 1182 to the electrode 1142.

Still referring to FIG. 11, at block 1202 the method 1200 includes advancing the first catheter 100 through a first blood vessel 1300 as illustrated in FIG. 13A. For example, a user may advance the first catheter 100 through the first blood vessel 1300 to a first treatment location so that the first treatment portion 130 is advanced to the first treatment location.

Referring again to FIG. 11, at block 1204 the method 1200 further includes advancing the second catheter 100′ through a second blood vessel 1302. For example, and as illustrated in FIG. 13A, the user may advance the second catheter 100′ through the second blood vessel 1302 to a second treatment location in the second vessel. It is noted that the first blood vessel 1300 and the second blood vessel 1302 may be adjacent vessels such as a vein and an artery, though vein to vein and artery to artery treatments are contemplated and possible.

At block 1206 the method 1200 may include aligning the first treatment portion 130 of the first catheter 100 with the second treatment portion 130′ of the second catheter 100′. As shown in FIG. 13B, as the second catheter 100′ approaches the treatment location, and as the first treatment portion 130 and the second treatment portion 130′ are substantially aligned (e.g., both longitudinally and rotationally), the first treatment portion 130 and the second treatment portion 130′ are drawn to one another by the interaction of the attraction of the one or more magnetic arrays 120 and 120′, and/or the one or more magnetic arrays 122 and 122′, of the first and second catheters 100 and 100′.

The magnetic attraction may be sufficient to draw the first blood vessel 1300 closer to the second blood vessel 1302. In some embodiments, the magnetic attraction may be sufficient to draw the first blood vessel 1300 into contact with the second blood vessel 1302, thereby pulling the first blood vessel 1300 and the second blood vessel 1302 into contact with one another. Regardless, once aligned, the first catheter 100 and the second catheter 100′ may coapt together and may sandwich tissue for the first vessel and the second vessel therebetween. In some embodiments, the method 1200 may further include confirming alignment of the first catheter 100 and the second catheter 100′ via fluoroscopy or other imaging techniques.

Referring again to FIG. 11, at block 1208 the method 1200 may further include, after alignment between the first treatment portion 130 and the second treatment portion 130′of the first catheter 100 and the second catheter 100′, performing a vascular treatment procedure to modify the first blood vessel 1300 and/or the second blood vessel 1302 at the first treatment location and/or the second treatment location. For example, the electrode 142 of the first treatment portion 130 may be activated by providing a supply of energy to the electrode 142 from an energy source (e.g., an RF generator or the like). The electrical circuit may be closed by the placement of a ground pad contacting the patient. Activation of the electrode 142 may ablate tissue of the first blood vessel 1300 and the second blood vessel 1302 to form a fistula 1304 between the first blood vessel 1300 and the second blood vessel 1302, as generally illustrated in FIG. 13C. Upon formation of the fistula 1304, the first catheter 100 and the second catheter 100′ may be withdrawn from the respective first blood vessel 1300 and the second blood vessel 1302.

As previously discussed, the catheters and sliding limit assemblies discussed herein may be used in any of the systems 10, as well as the second catheter 100′.

Embodiments can be described with reference to the following numerical clause:
1. A system for forming a fistula, the system comprising: a first catheter configured to be advanced in a first vessel, the first catheter comprising a first treatment portion defining a first treatment slot and an electrode advanceable from the first treatment slot, wherein a length of the first treatment slot is adjustable; and a second catheter configured to be advanced in a second vessel, the second catheter comprising a second treatment portion defining a second treatment slot configured to receive the electrode, wherein the length of the second treatment slot is adjustable or non-adjustable.
2. The system of any preceding clause, wherein the first treatment portion comprises an electrode housing comprising: a first portion; and a second portion, wherein: the first portion and the second portion together define the first treatment slot extending between a first end wall of the first portion and a second end wall of the second portion; and the first portion defines a receiving slot positioned within the first treatment portion; the second portion being slidably positioned within the receiving slot such that a distance between the first end wall and the second end wall is adjustable.
3. The system of any preceding clause, further comprising a sliding limit assembly, the sliding limit assembly limiting a distance the second portion travels relative to the first portion.
4. The system of any preceding clause, wherein the sliding limit assembly comprises one or more grooves and one or more pins positioned within and slidable along the one or more grooves.
5. The system of any preceding clause, wherein the sliding limit assembly comprises a plurality of ridges formed on one of the first portion or the second portion and one or more protrusions formed on the other of the first portion and the second portion.
6. The system of any preceding clause, further comprising a rubber gasket coupled to one of the first portion and the second portion and configured to at least partially resist sliding of the first portion relative to the second portion.
7. The system of any preceding clause, wherein the first treatment slot is substantially U-shaped.
8. The system of any preceding clause, wherein the electrode comprises a first electrode and a second electrode longitudinally offset from the first electrode.
9. The system of any preceding clause, wherein the first treatment portion comprises a cover which is slidable to adjust the length of the first treatment slot.
10. The system of any preceding clause, wherein the first treatment portion comprises a first portion and a second portion adjustably coupled to the first portion via an interlocking mechanism.
11. The system of any preceding clause, wherein the first portion is radially expandable and expands about insertion of the second portion within the first portion.
12. The system of any preceding clause, wherein the second portion is radially compressible and compresses upon insertion of the second portion within the first portion.
13. The system of any preceding clause, further comprising a sliding sleeve, wherein: the first portion and the second portion together define the first treatment slot extending between a first end wall of the first portion and a second end wall of the second portion; the first portion defines a receiving slot positioned within the first treatment portion; the second portion being slidably positioned within the receiving slot such that a distance between the first end wall and the second end wall is adjustable; the first portion comprises a protruding section that protrudes from an exterior and interior of the second portion; the sliding sleeve is positioned on the exterior of the first portion and is operable to slide over and contact the protruding section of the first portion; the interlocking mechanism is provided by the protruding section and the sliding sleeve.
14. The system of any preceding clause, wherein: the first portion and the second portion together define the first treatment slot extending between a first end wall of the first portion and a second end wall of the second portion; the first portion defines a receiving slot positioned within the first treatment portion; the second portion being slidably positioned within the receiving slot such that a distance between the first end wall and the second end wall is adjustable; the second portion comprises a protruding section that protrudes from an exterior of the second portion; the first portion is radially expandable such that the first portion radially expands and deforms around the protruding section of the second portion when the second portion is inserted into the first portion; and the interlocking mechanism is provided by the protruding section and the first portion.
15. The system of any preceding clause, wherein: the first portion and the second portion together define the first treatment slot extending between a first end wall of the first portion and a second end wall of the second portion; the first portion defines a receiving slot positioned within the first treatment portion; the second portion being slidably positioned within the receiving slot such that a distance between the first end wall and the second end wall is adjustable; the second portion comprises a plurality of bores extending through the second portion; the first portion comprises one or more depressible buttons; the one or more depressible buttons engage one or more of the plurality of bores to hold the first portion relative to the second portion; and the interlocking mechanism is provided by the one or more depressible buttons and the plurality of bores.
16. A catheter for forming a fistula, the catheter comprising: a catheter body; an electrode housing coupled to the catheter body, the electrode housing comprising: a first portion; a second portion, wherein: the first portion and the second portion together define a treatment region extending between a first end wall of the first portion and a second end wall of the second portion; the first portion defines a receiving slot positioned within the treatment region; and the second portion being slidably positioned within the receiving slot such that a distance between the first end wall and the second end wall is adjustable; and an electrode configured to protrude from the treatment region.
17. The catheter of any preceding clause, further comprising a sliding limit assembly, the sliding limit assembly limiting a distance the second portion travels relative to the first portion.
18. The catheter of any preceding clause, wherein the sliding limit assembly comprises one or more grooves and one or more pins positioned within and slidable along the one or more grooves.
19. The catheter of any preceding clause, wherein the sliding limit assembly comprises a plurality of ridges formed one of the first portion or the second portion and one or more protrusions formed on the other of the first portion and the second portion.
20. The catheter of any preceding clause, wherein the treatment region is substantially U-shaped.
21. The catheter of any preceding clause, wherein the electrode comprises a first electrode and a second electrode longitudinally offset from the first electrode.
22. The catheter of any preceding clause, further comprising a rubber gasket coupled to one of the first portion and the second portion and configured to at least partially resist sliding of the first portion relative to the second portion.
23. A catheter for forming a fistula, the catheter comprising: a treatment portion comprising a first portion and a second portion, the treatment portion defining a treatment slot, wherein the first portion is adjustable relative to the second portion to adjust a length of the treatment slot; an electrode configured to project from the treatment slot; and a sliding limit assembly, the sliding limit assembly limiting a distance the second portion travels relative to the first portion.
24. The catheter of any preceding clause, wherein the sliding limit assembly comprises a plurality of catches coupled to the first portion or the second portion.
25. The catheter of any preceding clause, wherein the sliding limit assembly comprises one or more pins slidably positioned within one or more grooves formed within one of the first portion and the second portion.
26. A system for forming a fistula, the system comprising the catheter according to any of clauses 16 to 25, the catheter configured to be advanced in a first vessel; and further comprising a second catheter configured to be advanced in a second vessel, the second catheter comprising a second treatment portion defining a second treatment slot configured to receive the electrode, wherein the length of the second treatment slot is adjustable or non-adjustable.

It should now be understood that embodiments of the present disclosure are directed to devices, systems, and methods for forming a fistula between two blood vessels. In some embodiments, the devices and methods may be used to form a fistula between two blood vessels. More particularly, a catheter may be placed in each of two adjacent blood vessels to form a fistula therebetween with the catheters. For example, in some embodiments, a catheter for endovascular treatment of a blood vessel includes a treatment portion, a proximal magnetic array positioned proximal to the treatment portion, and a distal magnetic array positioned distal to the treatment portion. Each of the proximal magnetic array and the distal magnetic array includes a first magnetic segment having a first polarity and a second magnetic segment having a second polarity opposite to the first polarity, the second magnetic segment is disposed adjacent to the treatment portion. The opposite poled magnetic segments may provide improved coaptation with a second catheter, as will be described in greater detail herein, and tactile feedback (e.g., vibration, snapping into place feeling, or the like) to ensure proper alignment and/or coaptation. These and additional features and benefits will be described in greater detail herein.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A system for forming a fistula, the system comprising:
a first catheter configured to be advanced in a first vessel, the first catheter comprising a first treatment portion defining a first treatment slot and an electrode advanceable from the first treatment slot, wherein a length of the first treatment slot is adjustable; and
a second catheter configured to be advanced in a second vessel, the second catheter comprising a second treatment portion defining a second treatment slot configured to receive the electrode, wherein the length of the second treatment slot is adjustable.

2. The system of claim 1, wherein the first treatment portion comprises an electrode housing comprising:
a first portion; and
a second portion, wherein:
the first portion and the second portion together define the first treatment slot extending between a first end wall of the first portion and a second end wall of the second portion; and
the first portion defines a receiving slot positioned within the first treatment portion;
the second portion being slidably positioned within the receiving slot such that a distance between the first end wall and the second end wall is adjustable.

3. The system of claim 2, further comprising a sliding limit assembly, the sliding limit assembly limiting a distance the second portion travels relative to the first portion.

4. The system of claim 3, wherein the sliding limit assembly comprises one or more slots and one or more pins positioned within and slidable along the one or more slots.

5. The system of claim 3, wherein the sliding limit assembly comprises a plurality of ridges formed on one of the first portion or the second portion and one or more protrusions formed on the other of the first portion and the second portion.

6. The system of claim 2, further comprising a gasket or o-ring coupled to one of the first portion and the second portion and configured to at least partially resist sliding of the first portion relative to the second portion.

7. The system of claim 1, wherein the first treatment slot is substantially U-shaped.

8. The system of claim 1, wherein the electrode comprises a first electrode and a second electrode longitudinally offset from the first electrode.

9. The system of claim 1, wherein the first treatment portion comprises a sliding cover which is slidable to adjust the length of the first treatment slot.

10. The system of claim 1, wherein the first treatment portion comprises a first portion and a second portion adjustably coupled to the first portion via an interlocking mechanism.

11. The system of claim 10, wherein the first portion is radially expandable and expands about insertion of the second portion within the first portion.

12. The system of claim 10, wherein the second portion is radially compressible and compresses upon insertion of the second portion within the first portion.

13. The system of claim 10, further comprising a sliding sleeve, wherein:
the first portion and the second portion together define the first treatment slot extending between a first end wall of the first portion and a second end wall of the second portion;
the first portion defines a receiving slot positioned within the first treatment portion;
the second portion being slidably positioned within the receiving slot such that a distance between the first end wall and the second end wall is adjustable;
the first portion comprises a protruding section that protrudes from an exterior and interior of the second portion;
the sliding sleeve is positioned on the exterior of the first portion and is operable to slide over and contact the protruding section of the first portion;
the interlocking mechanism is provided by the protruding section and the sliding sleeve.

14. The system of claim 10, wherein:
the first portion and the second portion together define the first treatment slot extending between a first end wall of the first portion and a second end wall of the second portion;
the first portion defines a receiving slot positioned within the first treatment portion;
the second portion being slidably positioned within the receiving slot such that a distance between the first end wall and the second end wall is adjustable;
the second portion comprises a protruding section that protrudes from an exterior of the second portion;
the first portion is radially expandable such that the first portion radially expands and deforms around the protruding section of the second portion when the second portion is inserted into the first portion; and
the interlocking mechanism is provided by the protruding section and the first portion.

15. The system of claim 10, wherein:
the first portion and the second portion together define the first treatment slot extending between a first end wall of the first portion and a second end wall of the second portion;
the first portion defines a receiving slot positioned within the first treatment portion;
the second portion being slidably positioned within the receiving slot such that a distance between the first end wall and the second end wall is adjustable;
the second portion comprises a plurality of bores extending through the second portion;
the first portion comprises one or more depressible buttons;
the one or more depressible buttons engage one or more of the plurality of bores to hold the first portion relative to the second portion; and
the interlocking mechanism is provided by the one or more depressible buttons and the plurality of bores.

16. A catheter for forming a fistula, the catheter comprising:
a catheter body;
an electrode housing coupled to the catheter body, the electrode housing comprising:
a first portion;
a second portion, wherein:
the first portion and the second portion together define a treatment region extending between a first end wall of the first portion and a second end wall of the second portion;
the first portion defines a receiving slot positioned within the treatment region; and
the second portion being slidably positioned within the receiving slot such that a distance between the first end wall and the second end wall is adjustable; and
an electrode configured to protrude from the treatment region.

17. The catheter of claim 16, further comprising a sliding limit assembly, the sliding limit assembly limiting a distance the second portion travels relative to the first portion.

18. The catheter of claim 17, wherein the sliding limit assembly comprises one or more slots and one or more pins positioned within and slidable along the one or more slots.

19. The catheter of claim 17, wherein the sliding limit assembly comprises a plurality of ridges formed on one of the first portion or the second portion and one or more protrusions formed on the other of the first portion and the second portion.

20. The catheter of claim 16, wherein the treatment region is substantially U-shaped.

21. The catheter of claim 16, wherein the electrode comprises a first electrode and a second electrode longitudinally offset from the first electrode.

22. The catheter of claim 16, further comprising a gasket or o-ring coupled to one of the first portion and the second portion and configured to at least partially resist sliding of the first portion relative to the second portion.

23. A catheter for forming a fistula, the catheter comprising:
a treatment portion comprising a first portion and a second portion, the treatment portion defining a treatment slot, wherein the first portion is adjustable relative to the second portion to adjust a length of the treatment slot;
an electrode configured to project from the treatment slot; and
a sliding limit assembly, the sliding limit assembly limiting a distance the second portion travels relative to the first portion.

24. The catheter of claim 23, wherein the sliding limit assembly comprises a plurality of catches coupled to the first portion or the second portion.

25. The catheter of claim 23, wherein the sliding limit assembly comprises one or more pins slidably positioned within one or more slots formed within one of the first portion and the second portion.
